(19) United States
(12) Patent Application Publication
(10) Pub. No.: US 2007/0270671 A1
(43) Pub. Date: Nov. 22, 2007

(54) PHYSIOLOGICAL SIGNAL PROCESSING DEVICES AND ASSOCIATED PROCESSING METHODS

(75) Inventor: Yoav Gal, Berkeley, CA (US)

Correspondence Address: WINSTON & STRAWN LLP;PATENT DEPARTMENT, 1700 K STREET, N.W., WASHINGTON, DC, 20006, US

(73) Assignee: VivoMetrics, Inc.

(21) Appl. No.: 11/733,046

(22) Filed: Apr. 9, 2007

Related U.S. Application Data
(60) Provisional application No. 60/791,095, filed on Apr. 10, 2006.

(51) Int. Cl. A61B 5/00

(52) U.S. Cl. 600/301

(57) ABSTRACT

The invention provides improved devices for processing data from one or more physiological sensors based on parallel processing. The provided devices are small, low power, and readily configurable for use in most physiological monitoring applications. In a preferred embodiment, the provided devices are used for ambulatory monitoring of a subject's cardio-respiratory systems, and in particular, process data from one or more respiratory inductive plethysmographic sensors.

### CROSS-REFERENCE TO RELATED APPLICATIONS

This application claims the benefit of U.S. provisional application No. 60/791,095 filed Apr. 10, 2006.

### FIELD OF THE INVENTION

The present invention concerns the field of physiological data collection and physiological monitoring in ambulatory settings, clinical settings, or hospital settings, and specifically provides small, low power, processing devices useful therein.

### BACKGROUND OF THE INVENTION

Physiological monitoring systems usually include processing devices and circuitry for filtering, processing, and analysis of sensor data. These devices are usually designed around a standard microprocessor or microcontroller that processes data in single-instruction-single-data (SISD) manner. Accordingly, processing tasks must be performed sequentially in time. Further, current physiological processing devices often allocate signal processing function to the microprocessor in order to minimize external circuitry. Such signal processing further contends for the microprocessor's sequential processing capacities.

For the above reasons, as the number of sensors increase and as sensor processing tasks become more complex, a physiological processing device's microprocessor or microcontroller can rapidly become a bottleneck. Adding further sensors or processing tasks can require device redesign, at least to include a more capable microprocessor. Even then, processing is sequential.

It is apparent that processing devices useful in physiological monitoring equipment that independently process data from independent sensors and that are scalable are desirable. Such devices would more readily accommodate additional sensors and more complex processing and additionally would permit real time response to multiple physiological data streams.

### SUMMARY OF THE INVENTION

This invention provides such improved processing devices for physiological monitoring equipment, especially for ambulatory monitoring equipment. In the following, the devices provided by this invention are often referred to equivalently as processing boards, printed circuit boards, PC boards, URB (standing for “upgraded respiration boards”) boards, and the like. Specifically, devices of the present invention are preferably configured as small, low power, self contained processing boards architected and designed to be useful in personal physiological data collection systems, especially in such systems intended for ambulatory use. The devices permit autonomous and ambulatory monitoring, easy integration into third-party monitoring systems, and ready accommodation of additional sensors or processing tasks.

Generally, URB boards include analog front end (“AFE”) circuits for processing and digitizing analog sensor signals and digital circuits for processing digitized sensor data (or data from sensors that directly provide digital data. The digital circuits are configured like multiple-instruction-multiple-data (MIMD) processing, and in various embodiments can have fixed configurations (as in a purpose-built integrated circuit), or can be configurable, e.g., by firmware programming that is initially-loaded upon start-up, as are FPGAs, or can be a parallel processor or processors configured by software loaded during running, or the like. The firmware configures the digital circuitry to, in parallel, receive sensor signals (after preprocessing by analog circuits if necessary), process the received signals, transform processed signals into physiologically meaningful data, and output into an encoded data stream. Optional capabilities can include hardware checking, sensor checking (e.g., range checking), power management (e.g., Li-ion battery management), and the like. Different embodiments of this invention can have physical sizes and power requirements optimized for specific physiological monitoring applications.

Preferably, MIMD circuitry of this invention are built in field-programmable gate arrays (“FPGA”) which can be firmware-configured into numerous independent functional units that process signals from various physiological sensors according to algorithms suitable to the individual sensors. Preferred FPGAs are selected from a family of low-power FPGA devices so that processing capability can be routinely adapted to application requirements. Preferred FPGA families include have low quiescent current, pin compatibility among family members, internal RAM both block and distributed, software support tools for mixed schematic, state machine, HDL design, and hierarchical macro based design (useful in this case as processing functions are often replicated as in digital filters, computational blocks, etc.). In other embodiments, digital signal processors, specifically configured integrated circuits, one or more parallel processors, and the like, can be used.

Configurable FPGA-based and similar architectures are preferred because they have advantages including: efficient use of power in that task-switch and other processor management overheads are obviated; independent processing of sensor data streams so that true real-time response to multiple physiological inputs is possible, e.g., real-time response to respiratory events, cardiac events, position and activity events, temperature deviations, and the like; reduced memory contention because memory can be distributed and replicated along with processing blocks. These advantages are not found in standard microprocessors which implement single-instruction-single-data processing and can be significantly limited by memory access.

In more detail, FPGA-based and similar parallel, configurable (and other parallel) architectures allow replication and expansion of processing functions with little or no impact on performance. Independent processing resources can be provided for each of several sensors (whether replicated or unique) so that processing of signals from one sensor does not impact processing of signals from other sensors. Furthermore, additional performance improvements are possible because individual processing sub-functions of each sensor processing function can be independently pipelined at full clock speed and without interruption. Such parallel function replication and pipelining is not possible with CPU based designs.

URB boards of this invention are preferably designed to receive and process signals from multiple physiological sensors including, in preferred embodiments, at least one inductive plethysmographic (“IP”) sensor. IP sensors generally include an elastic material with embedded conductive elements (further described in U.S. patent application Ser. No. 11/233,317, filed Sep. 21, 2005, which is included by reference herein it its entirety). When such sensors are applied to a subject, size changes due to physiological processes (e.g., respiratory or cardiac activity) cause stretching and contraction of the sensor which is translated into changes in one or more electrical properties (e.g., inductance) of the sensor's conductive elements. Such changes in electrical properties can be measured and processed to yield physiological information.

For example, respiration can be sensed when IP sensors are applied to a subject's chest and/or abdomen (“Respiration Inductive Plethysmography” or “RIP”) (see, e.g., U.S. Pat. No. 6,551,252, which is included by reference herein it its entirety). RIP sensors can be capable of measuring small changes in body-sizes, e.g., well below 1%. On the other hand, such sensors can be affected by extraneous factors above and beyond respiration, e.g., shock and vibration from walking running and jumping, speech and cough, mechanical vibration coupled from external sources etc. Therefore, preferred RIP processing circuitry (and processing circuitry for other sensors) performs the steps of: amplifying input RIP sensor signals without contributing noise (or without increasing the signal's dynamic range), calibrating amplified signals to fill available signal range (auto centering), filtering the signals to remove extraneous noise with minimal effect on respiration information (e.g., by a digital FIR filter), and analyzing the filtered signals to detect breathing preferably using one or more interrelated state machines (time domain and AGC analysis). However, in certain embodiments occurrences of individual breaths, individual coughs, periods of speech, and other respiratory-related events are important, and circuitry can be included to search for and recognize such respiratory-related events. Further, preferred RIP circuitry operates in parallel, can combine signals from multiple RIP sensors (e.g., chest and abdomen sensors), and automatically adjust to the number of sensors used, to the subject's size, and to the subject's activity level.

Devices of this invention also preferably process signals from other sensors, e.g., skin temperature sensors, skin conductance sensors, accelerometer sensors (preferably sensitive to three independent acceleration components (“3D”)), body core temperature sensors (e.g., capsule-like devices that transmit temperature information and are swallowed by a subject), and the like. One or more cardiac sensors are preferred, for example, sensors that transmit pulses upon sensing a heart beat (e.g., Polar Wearlink®), or one or more leads of ECG signals, IP thoracocardiogram sensors (see, e.g., U.S. Pat. No. 6,783,498, which is included by reference herein it its entirety), or other cardiac sensors. A device of this invention can also provide for receiving signals from further optional sensors, e.g., an optional arterial oxygenation sensor such as a pulse oximeter, which are then processed as necessary.

Preferred embodiments also include input-output ports for digital and analog data, e.g., standard SPI-16 (serial peripheral interface with 16 bit data packages) interface capabilities as are built in to many 3^{rd} party OEM systems, standard serial (or parallel) ports for receiving signals from further sensors, standard USB ports, and the like. A range of external connectors for input and output signals and for battery power facilitate further application adaptability.

Preferred embodiments also preferably include selected usability features. Power saving features for digital circuitry are known in the art and can be included. Power saving features for other circuitry can selectively power up sensors and sensor analog front-end (AFE') circuitry according to application, sensor presence, and sensor sampling rate. Also, when a low-battery condition is detected, further power saving features can disable higher performance processing circuitry or shut down less important sensors. Less important sensors vary from embodiment to embodiment and can include abdominal RIP sensors, accelerometers, temperature sensors, and the like. The URB can also continuously monitor sensor signals to determine conditions such as open/short circuit, out of range, excessive noise etc. In a preferred embodiment, results of such signal monitoring can be translated into one or more bits which are encoded with each transmission of physiological data from each sensor. Typical transmission rates are, e.g., 1 sec. for low definition (LD) data types, 50 msec for high definition (HD) respiration signals, the ECG sample rate for HD ECG, and so forth. With this scheme, configuration by an attached device can be obviated. Diagnostic facilities can be included which, e.g., monitor device test points, output raw sensor signals, circuitry temperatures, values of LED indicators, and so forth.

This invention also includes software products, e.g., FPGA configuration files (or other parallel software), implementing the methods of this invention. Also, the teachings of this invention can readily be implemented in physical configurations and arrangements known (or to be developed) in the electronics arts, and these alternative configurations and arrangements are part of the this invention.

In a preferred embodiment, this invention provides a device for processing signals from a plurality of physiological sensors having a plurality of functional digital, processing units that are configurable to perform steps including receiving signals from the sensors, processing the received signals to determine physiological information, and multiplexing the determined physiological information in one or more output signals, wherein, for the signals for two or more sensors, the steps of receiving and processing are performed by one or more processing units that operate concurrently and in parallel.

Aspect of the embodiments of this invention include that, for one or more sensor signals, two or more of the associated processing units are operate in series as a processing pipeline; and that separate physiological information is derived from two or more sensor signals in a substantially simultaneous manner; and that the device further includes a field programmable gate array (FPGA); and that the functional digital, processing units are configured by loading firmware prior to the sensor signal processing; and that the device further includes analog front end (AFE) circuitry for analog preprocessing of signals from analog sensors; and that some or all of the circuitry for processing a particular sensor signal is powered down if a signal is not being received from the particular sensor; and that some or all of the circuitry for processing a particular sensor signal is powered down for some or all of the time interval between signal samples received from the particular sensor; and that the functional digital, processing units are further configurable to process a signal from at least one of an accelerometer sensor, an ECG sensor, a heart rate sensor, a body temperature sensor, an electroencephalographic sensor, and a sound sensor; and that the device further includes one or more sensors sensitive to status of the device; and wherein the output signal further includes device status information determined from the device sensors; and that the concurrent processing further includes determining status of signals from one or more sensors; and wherein the output signal further includes the determined sensor-signal status information.

In another preferred embodiment, the invention provides a method for processing signals from a plurality of physiological sensors including receiving concurrently signals from two or more of the sensors receiving signals from the sensors, processing the received signals to determine physiological information, and multiplexing the determined physiological information in one or more output signals, wherein the receiving and processing proceed concurrently and in parallel for the signals from two or more of the sensors.

Aspect of the embodiments of this invention include that, for one or more sensor signals, concurrent processing includes two or more steps arranged in series and occurring simultaneously as a processing pipeline; and that processing of at least one sensor signal does not substantially delay processing of other sensor signals; and that the method further includes receiving signals sensitive to status of the device; and wherein the output signal further includes device status information; and that the sensor includes one or more of an inductive plethysmographic (IP) sensor, a respiratory IP (RIP) sensor, an accelerometer sensor, an ECG sensor, a heart rate sensor, a body temperature sensor, an electroencephalographic sensor, and a sound sensor; and that the method further includes adjusting the processing of one or more sensor signals according to calibration information, the calibration information being determined during a previous calibration period prior the processing; and that the method further includes determining the calibration information for two or more sensors during the same calibration period; and that the output signal includes one of more calibrated sensor signals; and that the calibration information for one or more IP sensors includes a signal output range, and wherein the IP sensor signals are centered within the output range; and that the calibration information for one or more accelerometer sensors includes a vertical reference value; and that the method further includes receiving signals from two or more IP sensors sensitive to the subject's respiration (RIP), wherein respiratory information concerning the subject is determined in part by combining the RIP signals.

In another embodiment, the invention provides a system for physiological monitoring of a subject including a plurality of physiological sensors including one or more inductive plethysmographic (IP) sensor, and a device for processing signals from the plurality of sensors including a plurality of functional digital, processing units that are configurable to perform steps including receiving signals from the IP sensor and at one or more other sensor, processing the received signals to determine physiological information concerning the subject, and multiplexing the determined physiological information in one or more output signals, wherein, for the signals for the IP sensor and one or more other sensors, the steps of receiving and processing are performed by one or more processing units that operate concurrently and in parallel.

Aspect of the embodiments of this invention include that the system further includes one or more sensors selected from an accelerometer sensor, an ECG sensor, a heart rate sensor, a body temperature sensor, an electroencephalographic sensor, and a sound sensor wherein the functional digital, processing units are configurable to process signals from the one or more sensors; and that the system further includes a plurality of IP sensors sensitive to the subject's respiration (RIP sensor), and wherein the concurrent processing further includes combining signals from at least two RIP sensors to determine respiratory information concerning the subject; and that the system further includes a housing, or a battery for powering the device, or a wireless communication unit for wirelessly communicating the physiological information remotely from the system, or a data recording unit for recording at recording the physiological information locally at the system

This invention is suitable for monitoring a mammal such as a human, horse, monkey, dog, cat, cattle, and so forth.

Further aspects and details and alternate combinations of the elements of this invention will be apparent from the following detailed description and are also within the scope of the inventor's invention.

### BRIEF DESCRIPTION OF THE DRAWINGS

The present invention may be understood more fully by reference to the following detailed description of the preferred embodiment of the present invention, illustrative examples of specific embodiments of the invention and the appended figures in which:

FIG. 1 illustrates exemplary systems including the processing device of this invention;

FIG. 2 illustrates architecture of the processing device of this invention;

FIG. 3 illustrates exemplary architecture for respiration sensor processing;

FIGS. 4A-C illustrates exemplary recursive hardware processing for RIP sensors;

FIG. 5 illustrates exemplary graphic display of HD RIP output;

FIGS. 6A-C illustrates exemplary physical arrangements of the processing device of this invention; and

FIG. 7A-B illustrate exemplary physical arrangements of a processing board;

### DETAIL DESCRIPTION OF THE PREFERRED EMBODIMENTS

This section describes preferred devices of this invention: their application in physiological monitoring systems, their processing organizations and methods, and their physical and hardware configuration. In the following, (and in the application as a whole), headings and legends are used for clarity and convenience only.

Applications of Devices of this Invention

The processing devices and boards of this invention are useful components in physiological monitoring systems directed to a number of different monitoring applications, e.g., monitoring of an ambulatory subject, patient monitoring in the clinic or the hospital, physiological and medical research, pharmaceutical evaluation, and the like. Specifically, ambulatory applications include monitoring for disease and disease treatment, monitoring of firefighters, soldiers, and other similarly placed subjects, monitoring in connection with athletics and physical training, and so forth. Although described herein with respect to human monitoring applications, devices of this invention are also useful for monitoring animals, e.g., in veterinary applications.

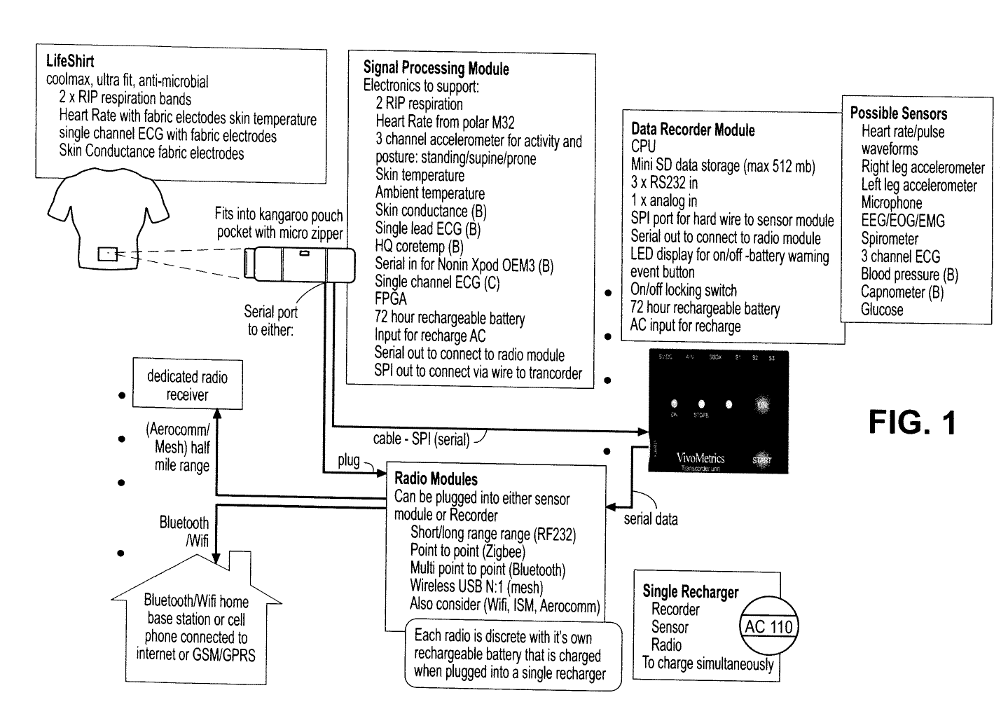

FIG. 1 illustrates an exemplary application of a device of this invention in an exemplary ambulatory physiological monitoring system. The illustrated system includes a number of individually packaged function units. A monitoring sensor subsystem is configured as a monitoring garment into which physiological sensors are incorporated or on which sensor are mounted or carried. The illustrated monitoring subsystem is configured as a shirt-like garment, but can also be configured as a band, a belt, a hat, a shoe, and so forth. An example of a band-like monitoring system is described in U.S. provisional patent application Ser. No. 60/730,980 filed Oct. 26, 2005.

A sensor signal processing subsystem receives raw sensor signals and processes them using a processing device of this invention. Preferably, devices of this invention multiples processed sensor signals into a serial digital output data stream. Processed data can be either stored at the monitored subject or transmitted remotely from the monitored subject or both. For ambulatory subjects, remote transmission is preferably wireless; for subject in the clinic and hospital a wired connection may be sufficient. In the illustrated embodiment, a communications subsystem comprises one or more wireless modules transmitting according to standard radio protocols. As illustrated, this device can also include a data recording and control subsystem including a data recorder module. This module can record processed sensors data for later use on flash memory cards or devices, or micro-hard-discs, or the like.

This system is illustrated in an embodiment in which the separate subsystems are separately packaged. This allows different monitoring systems to be readily assembled in the field from a single family of inter-communicating subsystem modules. For example, a complete monitoring system that transmits subject monitoring data in real time can be assembled from a monitoring-sensor subsystem, a sensor-signal-processing subsystem, and a communication subsystem. Alternately, the communications subsystem can be replaced by a data recording/control subsystem if subject monitoring data can be recorded for later use. Also alternately, a monitoring system can include both a communications subsystem and a data recording/control subsystem so that, e.g., summary monitoring data is available in real time while detailed monitoring data is available for later use.

As illustrated, the subsystems have separate physical packages; in other embodiments two or more subsystems can be packaged together leading to composite monitoring systems, e.g., as just described. Also as illustrated, the subsystems local to a monitored subject are linked by separate wires, cables, optical fibers, or the like; in other embodiments, personal a wired or wireless LAN may be used.

Processing Organization

The small, low power, processing devices according to this invention process of physiological sensor signals in a parallel and/or in a pipelined manner using digital circuits capable of concurrently executing multiple instructions or more generally, multiple Boolean and other functions on multiple data items. These processing capabilities are often referred to a “multiple-instruction-multiple-data” or MIMD. The words “concurrent task processing” are used in this context to refer to processing multiple tasks at each moment so that the tasks are processed at the same time. This is to be distinguished from processing by single instruction devices which achieve an illusion of concurrent task processing by processing at one time a portion of only a single task for a short time and then switching to processing a portion of another task; such task processing is not truly concurrent and tasks are not actually processed at the same time.

This invention can utilize MIMD digital circuitry of many architectures. Preferred MIMD circuitry is also readily configurable and re-configurable, has controllable low power requirements, has numerous input/output ports operating according to standard device protocols, is available in a single physical packaged, and so forth. A currently preferred implementation device has the architecture and properties of field programmable gate arrays (“FPGA”).

Since subsequent description is directed to such preferred FPGA implementations, FGPA architecture is very briefly described. An FPGA is an integrated circuit having at least several (and often a large number of) similar independent building blocks which can be configured together to perform many different processing functions. Usually, all building block circuits are capable of functioning at the same maximum clock speed (if so configured). FPGAs also have at least several (and often a large number of) of input/output pins or ports and associated drivers that can themselves be configured to variously connect to the building blocks. FPGAs are dynamically configured, usually during each power-up, by loading a bit string which controls the interconnection of building blocks among themselves and with the input/output ports for the duration of the current power-up.

It can be appreciated, therefore, that the building block circuits provided by a particular FPGA permit true MIMD implementation of parallel computing that can be configured to provide concurrent and pipelined processing of each of a plurality of sensor signals in the following manner. First, signals from each sensor are configured and assigned to building blocks and to input and output ports that are separate and that operate independently of the blocks and ports assigned to signals from the other sensors. Second, if processing of a sensor signal can be divided into sequential and independent tasks, each task can be assigned to a separate group of building blocks which are configured to be linked to the building blocks assigned to the prior and subsequent processing task. The words “pipelined task processing” are used in this context to refer to concurrently processing multiple portions of a single task. To be pipelined, a task must have two or more independent portions.

Different FPGAs can be preferable for a different embodiments. An FPGA preferred for a specific embodiment provides sufficient building block circuits and input/output ports so it can perform all digital processing. Preferred FPGAs also should posses RAM blocks (useful in low power FIR filter implementation, translation tables, etc.), and permit low power operation. A preferred family of FPGAs is used in the exemplary embodiment is the “Spartan 2” family produced by Xilinx.

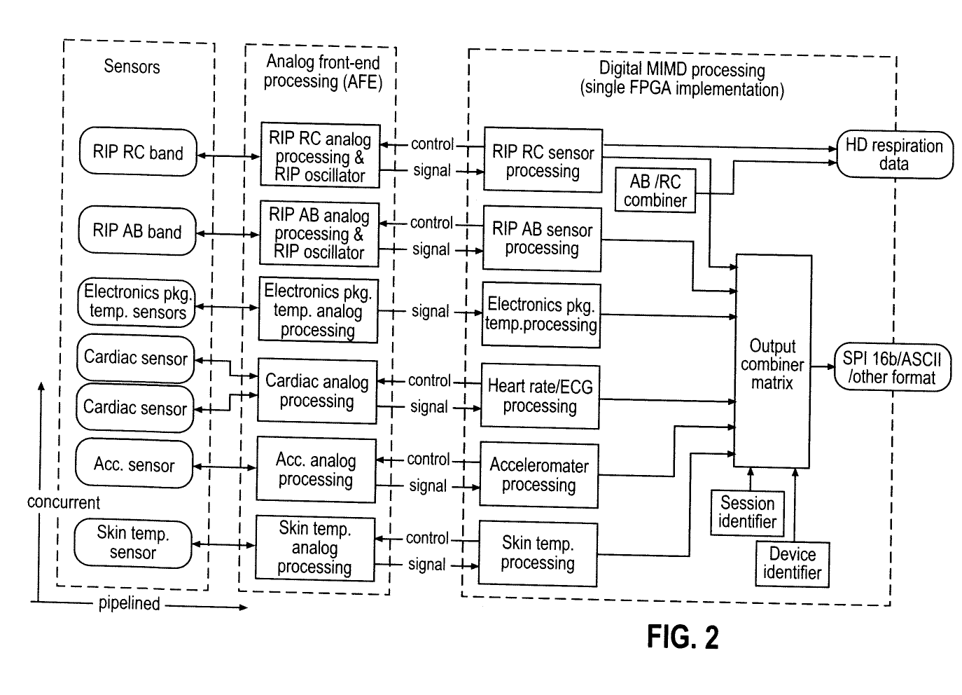

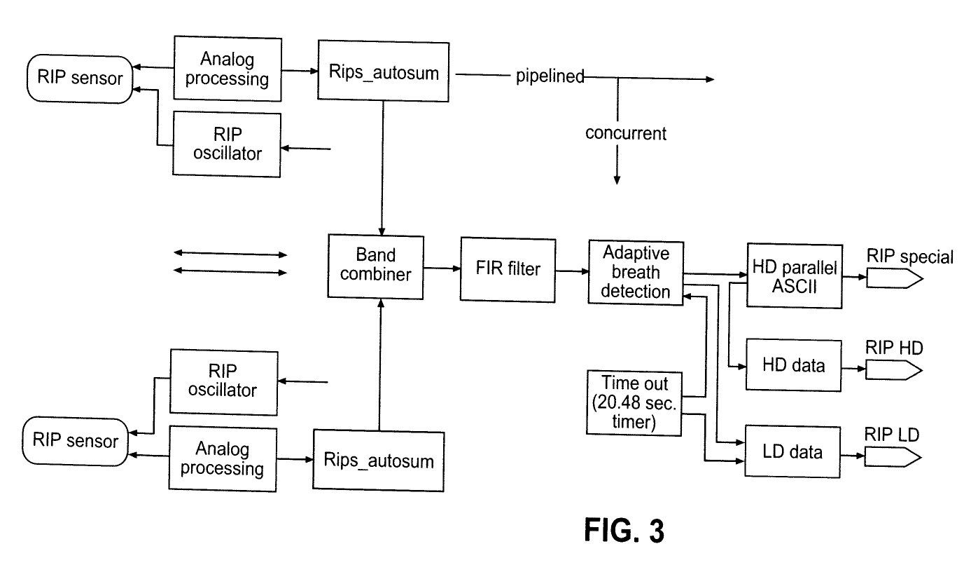

FIGS. 2 and 3 illustrate the high-level design (architecture) of an exemplary processing device of this invention directed primarily to cardio-respiratory and related physiological monitoring. This exemplary device is suitable for use in the system described with reference to FIG. 1. FIG. 2 is arranged, as indicated by the arrow legend, so that concurrent processing is stacked vertically and pipelined processing extends horizontally. Thus, the signal from each physiological sensor signal is processed from signal detection to signal output concurrently with the signals from other sensors in three sequential stages. Also, the processing of each physiological sensor signal is pipelined into three general stages: detection by the sensor, preprocessing by analog front-end circuitry (“AFE”), and digital domain processing by a single FPGA.

AFE refers to circuitry outside of the FPGA which excites a sensor (e.g., an oscillator for IP sensors; a pickup coil for Polar Wearlink type devices, and so forth); which performs analog filtering if necessary (e.g., for ECG and heart rate signals); and which converts signals to digital domain if necessary (e.g., for ECG, battery level sensing, heart rate signal pickup, 3D accelerometer channels). AFE's are designed to be low power, and where possible, to be selectively turned off by the FPGA to further reduce power draw.

AFE circuits are so designed that each sensor signal can be independently processed. Generally, these circuits perform filtering, normalization, and the like known to be necessary to minimize artifacts upon digital conversion and further processing inside the FPGA. Each sensor generally has a uniquely designed AFE; certain duplicated sensors (e.g., sensors for two or more RIP bands) can have replicated AFE's. Most AFE's receive control input (“cntl”) from the FPGA, which for example, turn on AFE power, select AFE parameters, and so forth.

For example, power to the 3D accelerometer is controlled by the FPGA controls so it is only active when needed (16 measures per second, using 8 mSec per measure results in ⅛^{th} of the power draw when compared to an AFE that is always ON). The FPGA also controls the output range of the accelerometer AFE so it can adapt to the physical activity level. Finally, the FPGA circuit self calibrates on power-up, resulting in relative insensitivity of processed accelerometer data to device placement on the body when measuring body position. Circuits (including the AFEs and FPGA) include self validation or “confidence values” derived in real time.

The AFE processing for each sensor's data is designed with one or more test points for validating proper functionality (e.g. disconnected or non functional sensor, out of range sensor operation, electrical noise presence in sensor data). Similar FPGA circuits are included to validate time domain characteristics of data derived from sensors. The resulting validation data (CV value for respiration, noise presence for HR) is embedded in the overall URB data stream enabling an external host system to use such data in view of its likely validity as best fits an application.

The embodiment illustrated, where concurrent processing is indicated vertically and pipeline processing with FPGA is indicated horizontally, performs digital processing for the illustrated sensors in a single FPGA. As illustrated, processing of each independent sensor is assigned to independent and concurrently functioning FPGA building blocks which receive sensor input signals from the AFE over dedicate input pins or ports. Where implemented processing methods for a sensor include multiple processing steps, the assigned building blocks are arranged in a sequential pipeline so that sequential processing steps can be concurrently performed on sequential input data.

Where sensors are duplicated, as with the illustrated RIP sensors, separate and independent pipelines are preferably assigned. Two similarly structured pipelines are assigned and implemented to process signals from the RC (rib cage) and AB (abdominal) RIP sensors. These blocks are fully parallel, perform the same function on incoming data, and do not detrimentally (in time or resource contention) impact each other processing or the processing of other sensors.

The embodiment illustrated preferably outputs one data stream containing all processed sensor data using a format suitable for reception by small electronic devices. Suitable standards are serial and parallel configurations using ASCII encoding. Suitable hardware standards include UART protocols (direct, RF, and USB), and SPI protocols and so forth. The output bus preferred in this embodiment format is serial peripheral interface configured to exchange 16 bit data packets (SPI-16). SPI-16 ports are built into many current FPGAs. Other outputs can optionally also be provided. Illustrated is ASCII-formatted, serial output of high definition (“HD”) respiratory data. In more detail, an output combiner matrix receives and buffers the results of sensor signal processing, and encodes the data into 16 bit self-defining data packets. Each packet includes a header identifying the data payload. Specific exemplary formats are described in more detail subsequently.

Individual FPGA building blocks are configured and interconnected according to a further design concept referred to herein as “recursive hardware processing”. Processing algorithms and processed data are adapted and are modified in real time according to interrelated rule sets. Briefly, during real-time sensor signal processing, multiple FPGA building block circuits measure different time domain and value domain parameters of the signal being analyzed and then pass these parameters to upstream or downstream FPGA circuitry which responds to the provided parameters by altering processing characteristics as dictated by embedded rules. These parameters are preferably measured by state machines and similar processing constructs configured from the FPGA circuitry.

Exemplary FPGA processing blocks and exemplary functional distribution within the FPGA are described in more detail in Tables 1 and 2.

TABLE 1
Input/output/processing of exemplary FPGA processing blocks

| Title | Inputs | Outputs | Processing |
|---|---|---|---|
| Top level - | all | host port - ascii & | receive raw sensor |
| | | spi | data and output |
| | | | formatted signals to |
| | | | host |
| C matrix | RIP HD & LD | RS232 & SPI16 | a data combiner |
| | RR latch | to external | matrix″ - formats and |
| | skin temp | modules | packetizes output data |
| | position & activity | | |
| | LD ambtemp LD | | |
| ASCII w16 - | parallel signal | ascii formatted | 16 bit ascii conversion |
| | | serial signal | and transmission; |
| | | | space mapping |
| Spi6_OD | parallel signal | spi16 formatted | 16 bit spi conversion |
| | | serial signal | SPI; bus master in |
| | | | mode 0″ |
| Respiration #2 | variable-frequency | RIP HD & LD | processing for one |
| | plethysmographic | digital signals | RIP sensor |
| | oscillator | | |
| RIP auto sum | variable-frequency | digitized | converts analog |
| | plethysmographic | plethysmographic | oscillator output to |
| | oscillator | signal | digital pulse sequence; |
| | | pulse counts | counts pulses |
| One RIP auto | | | “HIRES BAND CTR - |
| | | | dual modulus RIP |
| | | | band counter” |
| Fig filt 255 | digitized oscillator | filtered digitized | 256 tap FIR filter |
| | pulses | oscillator pulses | filter characteristics |
| Store - fir2 | | sequences of | coefficients and data |
| | | signed coefficient | from internal storage |
| | | & data 16 bit | |
| | | values | |
| ipmac 36 | sequences of | filtered data | 36 bit signed multiply |
| | signed coefficient | | and add; output |
| | & data | | justified to 16 bit; |
| | | | coefficient range 64K |
| | | | (0.0625 (1/16th)) |
| mull 16_ser | | | unsigned 16 × 16 |
| | | | serial multiplier |
| BDL-ADCV | | | “Breath Detector |
| | | | Logic - Adaptive |
| | | | MVC” |
| nbrval | | | “generate trend (sign, |
| | | | value) using N 20 |
| | | | msec samples” |
| min-max values | | | “windowed swing |
| | | | detector” |

In this table the following abbreviations are used: LD=low definition; hrave4=heart rate average; HD=high definition; ambtemp=temp of URB board; RR=respiration rate; HR LD (average 4)=low definition average of last 4 ECG RR intervals; MAC=multiply and accumulate.
Adaptive Power Handling

Adaptive power control is advantageous for battery-powered portable applications of the processing devices of this invention. Basically, adaptive power consumption turns-off or decreases power to hardware components, e.g., sensors, supporting analog circuits, and FPGA processing blocks, whenever possible. Embodiments of this invention can include one or more of the following features that achieve such power control.

First, devices of this invention can provide input ports for sensors that may be temporarily absent during a particular monitoring period because, for example, they are absent from a particular monitoring garment (or subsystem), or are not connected to their input ports, or have failed. Also various sensors may be permanently absent from an embodiment. Devices of this invention preferably sense absence of sensor input or aberrant sensor input and power down associated AFE circuits and FPGA functions.

Further, certain sensors may be sampled at sufficiently low rates that they and their processing circuits have no useful function for a significant fraction of a monitoring period. For example, accelerometers are sampled at 16 Hz; and temperatures are sampled at 10 Hz. Power is therefore supplied to accelerometers and to their associated AFE circuits for approximately 8 mSec at every approximately 64 mSec sampling time (or as otherwise convenient); for temperature sensors, power can be applied for approximately 8 mSec at every approximately 100 mSec sampling time (or as otherwise convenient). Thus, power requirements for these sensors, and for other sensors sampled at similar rates, are cut to only 10-15% without impacting output data of the amount these sensors and circuits would require if they were continuously powered.

However, other sensors are sampled at higher rates and such power-saving strategies may need to be more refined. For example, IP respiration sensors have a variable frequency oscillator that is sampled at 50 Hz. A useful power saving strategy it to continuously power the IP oscillator but power the associated sampling circuits only every 20 mSec. Since heart rate and ECG leads are often sampled at 1 KHz or greater, periodic powering-down may not be possible.

Also, FPGA power can be adaptively controlled so that FPGA processing blocks are powered only when there is data to be processed. For example, FPGA blocks processing accelerometer, temperature, or RIP signals need be powered only when the associated AFE circuitry is powered. Adaptive power control of FPGA blocks can be achieved by gated clocking, which turns off the clock to circuits when not needed. Additionally, the power requirements of FPGA blocks depends directly on the applied logic clock speed. Power can be saved by supplying processing blocks with light computational loads with lower rate clock signals. Also, in certain embodiments, a very low-power standby mode can be implemented. All sensors and AFE circuits are powered down, and the only powered FPGA blocks are those circuits essential to resuming operations. Also, in certain embodiments, an externally writeable control register is provided so that device operations and power requirements can be externally controlled. Different bits in the control register power on or off different portions of device circuitry and/or different FPGA processing blocks.

For example, because of the above adaptive power controls, the exemplary embodiment can operate 200 or more hours on a single charge of a Lithium-ion battery having about 2000 mAmp/hours (with a range of preferably 1500-2500 mAmp/hours.

Prior to beginning operation, devices of this invention usually perform specific power up sequences. Although details of power up sequences are implementation dependent, most include the following phases. A short, e.g., 100 μSec., stage of high current draw, e.g., 100 mAmp. to several Amp. during which operating voltages are established throughout the device. Before operation, a configuration bit string must be stored in the SRAM control registers (via known interfaces, e.g., JTAG); characteristics of this phase depend on FPGA size and resources used but typically requires 100-200 mSec. during which 20-50 mAmp. of current is drawn. After configuration, a device may begin operation. For example, the exemplary device as described and illustrated herein (including sensor current draw) typically requires 5-10 mAmp (average 8 mAmp.) during routine operation.

Further, devices of this invention preferably provide verification and test points and outputs (including immediate visual output, e.g., LED outputs). Preferred test points permit profiling of power consumption, testing of firmware functioning and other programming, monitoring of sensor signal inputs, monitoring of key sensor signal processing events, and the like. An example of the latter, which is further described subsequently, are test points for profiling of IP, e.g., RIP, sensor functioning and output of waveforms and data generated during RIP processing. Test points (and LED outputs) are preferably defined by firmware.

Processing of Respiration IP Sensor Signals

Inductive plethysmography (“IP”) sensors and processing, particularly as directed to respiratory monitoring (“RIP”), is described with respect to FIG. 3. An IP sensor is generally linear, flexible and lengthwise expandable to be worn and move with a body part being monitored, e.g., a portion of a subject's chest in the case of RIP. The IP sensor includes an electrical conductor that has a special spatial configuration so that it produces a substantially linearly changing inductance across a working range of lengths. The conductor component of an IP sensor is part of an oscillator that, thereby, has a frequency varying in dependence on the expansion of the IP sensor.

The RIP sensor processing of this invention implements a low power oscillator that is modified version of LRC multivibrator and symmetrical comparator to convert a variable floating low amplitude sine wave to a digital (LVTTL) signal. The oscillator uses switching N channel FETs requiring very low voltage to turn on (VGS). The primary balanced winding of a pulse transformer is used as symmetrical load to both multivibrator FETs, and the RIP band itself is connected across the secondary of that transformer. The inductance changes of the band affects both FET loads resulting in symmetrical excitation to the gates, two sine waves with 180 degree phase shift appear at the gates, and no duty cycle modulation is observed. Proper component selection and pulse transformer design result in well below 1 mA current need to run the full RIP AFE. This oscillator requires at most 800 microAmp or less and produces a variable frequency output sufficiently free of noise and artifact that breath cycles can be reliably detected down to an amplitude of 20 out of 8192 (arbitrary inductance units).

What is measured is the output frequency of the RIP oscillator. The data is sampled at 50 Hz. The input circuit starts by counting the number of cycles seen in the recent pre-selected sample window to form the RIP data on which all processing is done. This step amplifies the change measured. The typical RIP cycle of 3.0 uSec results in 0.66% modulation in direct measurement. The processing range selected is 13 bits allowing an 8192 range for RIP data. Since the band changes inductance (changing oscillator frequency) according to it's stretch, each person/band combination results in a different RIP value. A centering circuit “oneRIP_auto” biases the RIP input word at a selected percentage of total range a short time after power up, or after a band went from being “disconnected/non operational/out of range” to “normal”. Output from two of these circuits is added to form one RIP value that is centered in the full range as seen in “rips_autosum”. This insures maximum possible fluctuation of data without range rollover events.

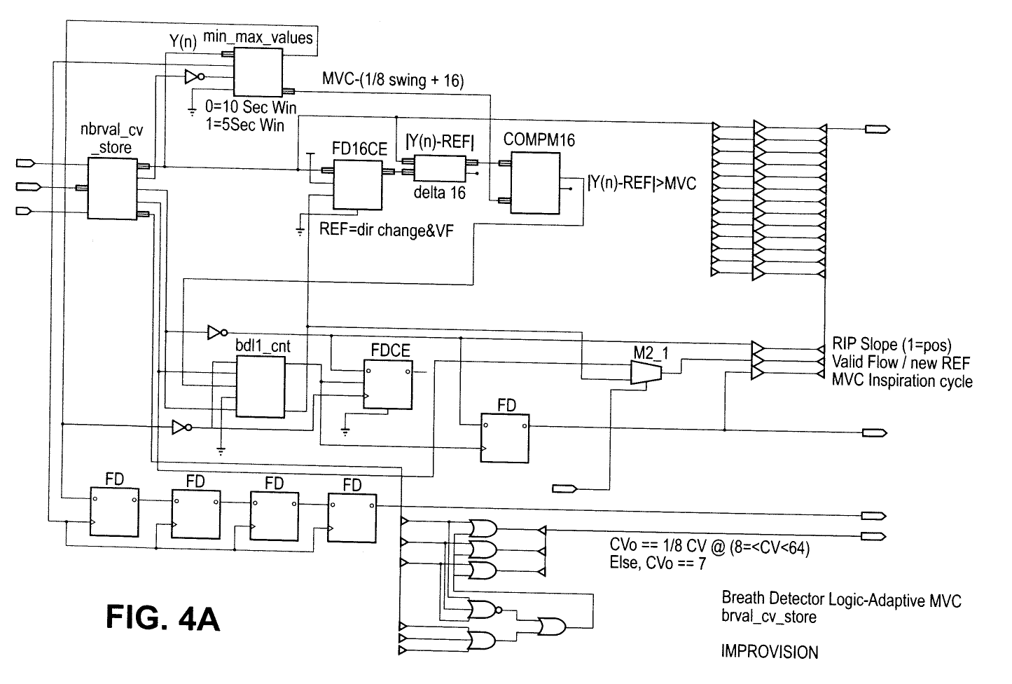

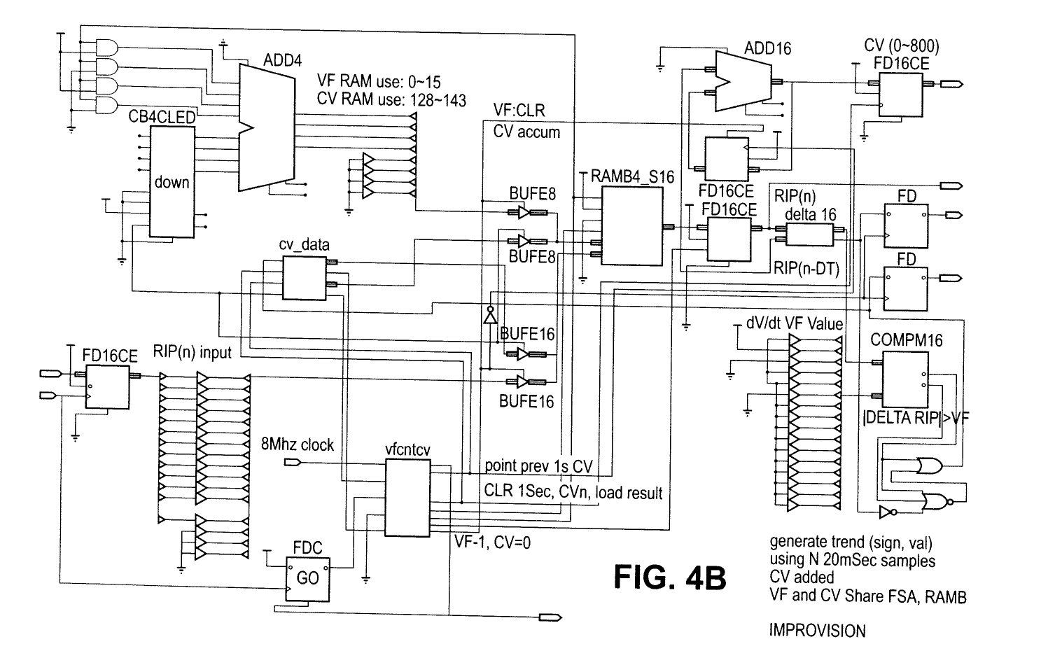

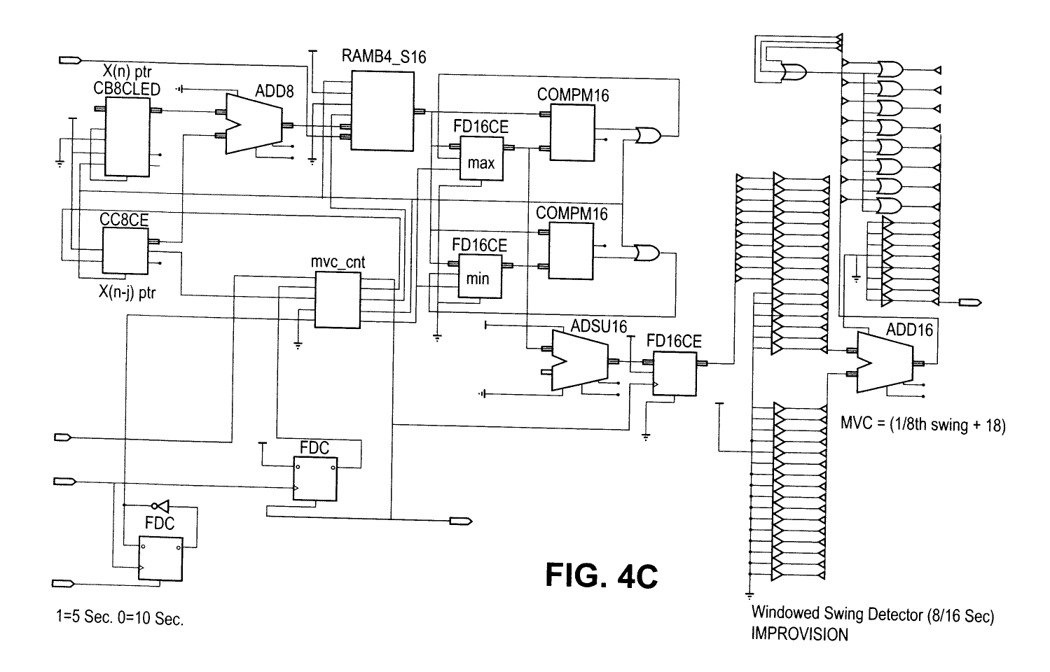

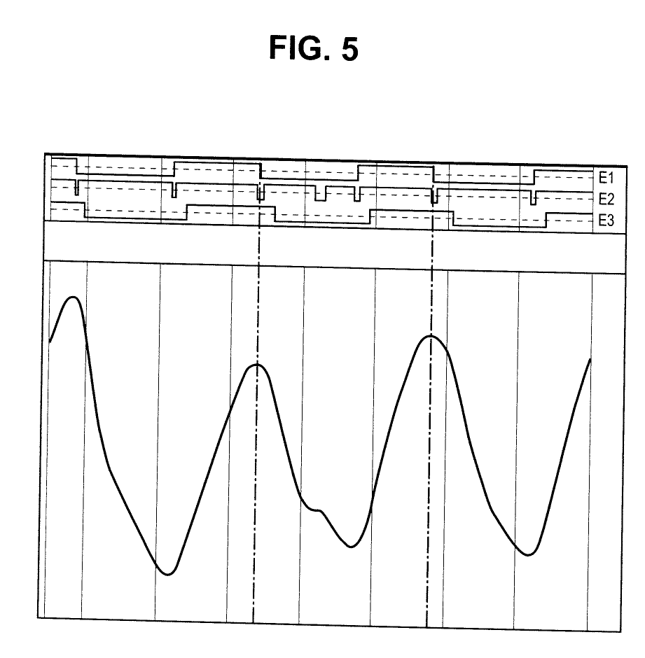

The combined RIP data (boxcar averaged and 20 mSec integrated) is next subjected to a low pass FIR filter “FIR_filt255“. 255 taps are used with a very sharp transition band matched to 75 BPM resulting in a very smooth high sensitivity RIP value HD waveform as seen in FIG. 5 main window. Breath cycle detection is made by “BDL_ADCVo” seen in FIGS. 4A-C.

The LD result of the above is an instantaneous respiration cycle in units of 20 mSec. This is next input into “RR_to_BPM” which counts the actual cycles over a moving 1 min. window to produce a results in “breaths per minute” units. This circuit considers 20.48 Sec. as a single breath cycle timeout and marks it accordingly resulting in very accurate respiration reading in the 3˜75 BPM range suitable for humans. Values of these parameters of course need to be appropriately chosen for animal monitoring (animals including monkeys, dogs, cats, horses, cattle, and similar mammals).

Output from a typical RIP sensor displays a small range of inductance change (less 1%, and less than 0.5%, and even smaller) and can contain artifacts due mechanical bodily changes and movements (shock and vibration from walking running and jumping, speech and cough, mechanical vibration coupled from external sources etc) in addition to the respiratory signal of interest. The RIP circuitry of this invention implements the following function with full application flexibility. Importantly, this circuitry automatically adjusts to the subject size, to the number of sensors used, and to the person's activity level.

The RIP processing circuits of this invention: amplify the input signal without contributing noise (or without increasing the signal's dynamic range); calibrate the signal as to maximize the use of available signal range (auto centering function), filter the signal to remove noise without effecting the respiration signal (digital FIR filter function); and analyze the signal (time domain and AGC analysis functions) to detect breath cycles. A set of interrelated state machines preferably perform the latter function. Further advantage is possible by processing in parallel multiple sensors (chest and abdomen).

As apparent, the respiration processing circuits process data in real time, continuously receiving RIP sensor input and continuously producing processed output values. However, there is an approximately 2.6 sec “pipeline” delay between receiving a data event and outputting a corresponding processed value. The FIR filter contributes the greatest portion of this pipeline delay

RIP oscillator output varies according to the particular sensor band: its fabrication, its average active length when worn, and other factors. To correct for these variations, on power up and on sensor band reconnection with power on, the preferred respiratory processing automatically centers output within output range. Preferably, output is centered at an output reading of approximately 25% (or 15%, or 20%, or 30%, or 35%) of its output dynamic range of 8192 counts. Also, the output from a sensor band that fails, is disconnected or is not within anticipated specification will be reset to this default value.

Using automatic centering and calibration, a device of this invention can process signals from one or more RIP sensors, e.g., rib cage and abdominal sensors, with separate processing circuitry for each sensor, and produce consistent and combinable output for all sensors.

On power up, automatic centering and calibration require approximately 5 Sec. to reach stable, centered, and validated RIP output values. Before power up, a monitored subject should don the RIP sensors, as by wearing a garment in which the sensor are embedded.

Several internal parameters are important in allowing processing devices of this invention to adapt to RIP sensor bands of various types, various active lengths, various mechanical constructions and constraints, etc. These parameters include the following:

first rank rip counter window: input boxcar counter measuring window; the limit value used detects a band that is not running fast enough and is thus considered to be disconnected, too stretched, poorly made, or having a failed auto center function;

VF:RIP: valid flow expressed in absolute filtered RIP value change over a selected time interval;

MVC: parameter of the cycle detector state machine used to track RIP HD values;

CV: confidence value

The “first rank RIP counter window” parameter1 is preferably optimized for a specific RIP sensor band whenever the band is integrated and electrically terminated by the user or a third party. The “RIP auto center” and “VF:RIP” parameters are set for the preferred overall activity range; they may also be used to set sensor output to a low activity or to a high activity range. Specifically, the “RIP auto center” parameter is the power-up and band hot-reconnect value.

The “MVC” parameters control signal jiggle discrimination. For example, external mechanical constraints placed on a RIP sensor band may induce larger than normal jiggles, and these parameters can be tuned accordingly. Specifically, the “MVC fixed bias” parameter has a preferred value of 16.

The “CV” parameters can be adjusted to fit low frequency environmental, e.g., mechanical, noise which may falsely appear as valid breathing patterns. The “CV” is preferably used to indicate the confidence level of this invention's breath cycle detector operation. Specifically, the “CV boxcar integrator length” parameter has a preferred value of 16 sec but may be set to other values; the “CV output translator” parameter has a preferred value of CVout=⅛^{th}CVin for 8=<CVin<64. Else, CVout=7 but may be set to other values. Finally, the “CV timeout window” parameter has a preferred value of 20.48 sec but may be set to other values greater than the “CV boxcar integrator length” parameter.

Preferred respiratory output includes either or both of higher definition (“HD”) of data values or lower definition derived respiratory parameters. The HD data output includes a sequence of values in the range of 0 to 8191 (arbitrary units) once every 20 mSec. These values represent a sensed current length of an IP sensor and, when the IP sensor is configured about the rib cage or abdomen of a monitored subject, the sensed length depends directly on a current respiratory volume. A system incorporating a device of this invention can make these values (and other output values) available in real-time to a monitored subject, monitoring personnel using wireless transmission or can store these values for later review by monitoring personnel using a data recorder module. These values can be displayed to visualize the monitored subject's breath waveforms.

A stand alone FPGA circuit (“min-max values”) determines the range of input data value change seen in the last 10 seconds and uses it to modify the “valid flow” value. Yet another circuit convolves the presence of “valid flow” with a 16 second window (also known as box car integration” operator) to indicate that the input data characteristics makes physiological sense or should be suspect of external mechanical disturbance.

Turning to FIG. 4A, the “min-max values” processing block feeds back to “nbrval_CV store” processing block when respiration flow in the last 10 seconds is below a certain level. Thereby, the way CV is derived is changed; the slope values used are changed; the resulting MVC value used in the RR cycle detection state machine is changed. This recursive feedback adapts the processing to “low and slow” chest movement typical of rest and sleep while rejecting “low and fast” activity that can be caused by mechanical vibration etc. The respiration cycle state machine uses “valid flow” measure (or time domain “slope”) to indicate possible inhalation/exhalation transitions.

Next, FIG. 4B illustrates that FPGA hardware-like parallel design can execute what would be cumbersome in a processor based design. The block RAM (“RAMB4_s16”) stores input RIP values and summed 1 sec accumulation of “valid flow” markers. The state machine “vfcntcv” manages the resources so that a running sum of the recent flow markers is maintained, and slope is calculated over a moving window. All calculations are done in less than 10 uSec. FIG. 4C illustrates the “min max values” processing block. Which stores a running list of the recent 256 consecutive or averaged RIP values (representing 5.12 or 10.24 seconds trace). The state machine “mvc_cnt” maintains the list, sorts the values for minima and maxima points, identify adjacent min max points and creates a value.

The above examples of computational blocks altering each other's input data and rule set is termed “recursive hardware processing” because it is functionally similar to software recursion but does so with minimal processing overhead since data are all operated on in real time. Thus, only one clock cycle is used for all data updates. Another example is the change of respiration MVC value according to moving box car histogram of the respiration data.

The device preferably outputs data in both HD and LD modes. All data is encoded using a uniform scheme. Data from all sensors in a specific application is sent in bursts. FIG. 3 shows that both bands combine automatically into one data stream. If a band is not present, or if a band is introduced—the auto-sum circuit will adjust and center the data in its range. Since one or more RIP sensors with both HD and LD information. two blocks are illustrated. The uniform encoding scheme permits mixing LD and HD data stream, but details are application specific. In certain applications, the HD and LD streams are separated; in other applications, HD and LD are combined on a single port

FIG. 5 illustrates such a display in which the bottom panel presents the waveforms of approximately three breaths of a seated subject. Signal values increase upward along the vertical axis and time increases rightward along the horizontal axis. It is apparent that this data is free of visually-apparent noise or artifact. In the sample screen below, 15 sec of data are displayed starting 4 sec into the trace. The red and blue markers are set at 9.76 and 14.56 sec respectively showing a 4.80 sec. time difference. At 10.72 seconds, the trace value is 3926 (mouse location when the screen was captured). The vertical scale is automatically set and min/max trace values (3636/4370) during the displayed interval displayed are shown on the right vertical bar. The upper panel presents three traces of binary indicators (value 0 or 1). The top trace, labeled “E1”, indicates the slope of the accompanying breath waveform, either upward during inhalation (value=1) or downward (value=0) during exhalation. The middle trace, labeled “E2”, indicates whether or not the data at the corresponding time is valid (value=1) or is not valid (value=0). And the bottom trace, labeled “E3”, indicates a detected respiratory cycle.

A first type of LD respiratory data is output upon recognition of a complete breath and includes breath duration expressed in units of 20 mSec. Termination of a current breath is recognized upon detection of inspiration marking the subsequent cycles. The cycle time is expressed in units of 20 mSec. If no valid breaths are detected within 20.48 sec, a unique word is transmitted. If two (or more) RIP sensor bands are present and active, e.g., a rib cage and an abdominal sensor band, breaths are recognized in an equally weighted sum of the output values of both sensor bands.

A second type of LD respiratory is output at 1 Hz data and includes respiration rate in breaths per minute (“BPM”) measured over a trailing 60 sec window by accumulated the number of complete breaths recognized (the first type of respiratory data). A confidence value accompanies this data and indicates whether or not RIP sensor data has been valid for the recent prior 16 sec period. If no breaths are recognized in three consecutive periods of duration 20.48 sec (respiratory time out periods), the BPM value to zero so the reported respiratory rate is updated and actually reflects the last 60 sec.

Processing of Accelerometer Sensor Signals

Preferred embodiments of the devices of this invention include accelerometers and accelerometer signal processing that provides position indication or activity indication or both. Miniaturized accelerometers are known in the art and include devices implemented as a micro electromechanical system (“MEMS”) and based on inertial or optical effects. A preferred accelerometer provides two or three dimensional acceleration signals (“2D” or “3D”) and is sized to fit as a component on a circuit board.

Accelerometer signals are sampled at sampling that are approximately a small multiple of the expected maximum mechanical frequency components produced by a monitored subject. A sampling rate of 16 Hz is preferred for most applications. A subject position signal reflects the maximum deviation from a vertical calibration of 1 sec. boxcar low pass (“LP”) filtered values of any accelerometer output channel. A vertical calibration is preferably the accelerometer orientation 8 sec. after initial power up (calibration period or window) (or when otherwise reset).

A subject activity level signal reflects a sum of 1 sec of Laplace high pass (“HP”) filtered values of all accelerometer output channels and is normalized to a range of 1 to 255 (arbitrary units). Optionally, the activity signal is interpreted and calibrated to pre-determined activity thresholds. Both the position and activity signals are LD signals typically transmitted at 1 Hz. Also, both the position and activity signals are preferably both derived in parallel from the sampled accelerometer channels.

Processing of Signals from Further Sensors

Preferred embodiments of the devices of this invention provide input ports and processing for cardiac signals from external cardiac sensors. External heart rate sensors can determine rate from vascular pulsations or cardiac electrical activity, for example, recognition of R waves in ECG signals, and can be directly or indirectly coupled to the devices of this invention.

A preferred external heart rate sensor is the Wearlink sensor produced by the Polar corporation. Upon recognition of a hear beat, a Wearlink sensor produces a coded electromagnetic burst which can be inductively received by a processing device. Burst timing is measured at 1 mSec resolution, and is output when received and a heart rate in beats per minute (“BPM”) is determined from a running average of the previous 8 inter-beat intervals and is output every 8 beats. If no heart beat is recognized for 4 sec., the BPM signal is set to zero. A flag accompanying this data indicates its validity. For example, if the BPM signal is set to 0, it is set to indicated that the data is invalid. Output signal is normalized to be from 1 to 255, where 1 represents a lowest heart rate and 255 represents a pre-selected maximum safe BPM value.

Optionally, one or more leads of ECG signals can be input and processed. This can supplement or replace an external heart rate sensor signal. The analog ECG signal is sampled at 1 kHz with 12 bit resolution. Each sample is preferably transmitted when available as HD ECG data. Alternately, cardiac parameters can be extracted from processed HD and transmitted at a lower rate. For example, R waves can be detected by known methods, and their occurrence times transmitted with approximately 1 mSec resolution. R wave occurrence times can be processed as above into a BPM signal and transmitted intermittently. Heart rate noise blocking and marking results in agile response (not averaging based) while removing data spikes caused by motion artifacts and electrode release. The output ECG data format is fully flexible in terms of data rate and resolution. This allows coarse to fine use.

Preferred embodiments of the devices of this invention provide input ports and processing for temperature signals from external temperature sensors. Skin temperature can be measured by thermistors. Thermistors are preferably sampled at approximately 10 Hz, and 10 samples are averages and transmitted at 1 Hz. Output temperature values are referenced to a 25° C. set point, have a range of 0 to 1023 with implied decimal point (physical range of 0˜102.3° C.), and have a worst case measurement accuracy of ±0.3° C. across the range from 10 to 60° C.

Body core temperature sensors are known in the form of ingestible capsules which, upon being swallowed, transmit, e.g., a signal with a frequency varying in dependence of sensed temperature. This frequency can be sensed by a processing device and transmitted at 1 Hz. Certain capsule sensors from HQ, Inc. transmit a frequency varying about a reference frequency of approximately 262 kHz. For such sensors, the transmitted values can reflect the difference between this reference frequency and the received frequency.

Also, skin conductance can be sensed, digitized and optionally ranged, or centered, or calibrated, and the like. Typically, transmission is at approximately 1 Hz. Sensors for thoracic skin conductance can also return ECG signals. Device status also can be sensed and transmitted, typically at 1 Hz. Status preferably includes device (or device PC board) temperature, battery level, and the like. Other sensors the processing of which can be included in various embodiments of this invention include one or more of the following: multiple accelerometers, such as right and left leg accelerometers; microphones and audio vibration sensors; electrodes for electroencephalograms, electro-oculograms, or electromyograms; spirometer; blood pressure; capnometer; glucose and other chemical sensors; and so forth. Also, it can be advantageous in certain embodiments to provide one or more pass-through ports for including signals from an external 3'rd party sensor in the output stream. In the case of devices producing digital signals, processing can be limited to packaging for output. In the case of devices producing analog signals, processing includes digitization and optionally ranged, or centered, or calibrated, and the like.

Generally, AFE circuitry or FPGA configuration can determine whether or not it is likely that a particular sensor is operational. Such an operational status signal can be used to control processing circuits for that sensor. For example, if the sensor is likely to be not operational, the related processing circuits are powered down, and can be powered again if the sensor resumes operation. Accordingly, sensor status received after power up (calibration period) can control the initial processing of a device of this invention. In this manner, a device can dynamically adapt to its sensor-signal input without the need for explicit control.

All the above sensor signals are preferably processed in parallel with other signals input to a device of this invention. Redundant sensors if present can be used (e.g., 2D and 3D accelerometers, polar and ECG HR derivation). This increases immunity to possible sensor failure or malfunction.

Output Processing

Processing devices according to this invention preferably multiplex processed sensor data into one or more serial output data streams. A single output data stream is preferred; multiple output data streams can be implemented according to application requirements. At lower network layers, output streams conform to one of the known serial protocols, such as those used in micro-controller or microcomputer applications. In the exemplary embodiment, multiplexed sensor output is available concurrently at a SPI-16 interface (e.g., with this device as a single master, 16 bit frames, mode 0, and rate of approximately one megabit per second) and at an ASCII formatted low voltage TTL (“LVTTL”) output.

At higher network layers, sensor data is formatted into self-defining data records (or data types), a unique type for each sensor. Table 2 illustrates record types defined for the exemplary embodiment of this invention. Preferably, record types are identified by unique bit strings in a field appearing at a fixed offset into each record. Records carrying low definition (LD) data from physiological sensors include at least the following two types. A first type of record is periodically transmitted and typically includes a parameter summarizing recent sensor outputs, e.g., recent heart rate in beats per minute. A second type of record is transmitted upon the occurrence of a particular physiological event and typically includes a descriptive parameter, e.g., for each heart beat or breath or cough or so forth, the time of its occurrence. In addition, to processing sensor data, records can also usefully include a field of one or more bits for sensor status. For example, an “OK” bit can indicate whether or not sensor processing found the associated data as likely to be valid or invalid.

TABLE 2
Exemplary record types and record formats
RIP bands, LD:

| | | | |
|---|---|---|---|
| OK | Data type | CV | instant inspiration cycle time |
| OK | Data type | CV | instant inspiration cycle time |

Skin temp sensor, LD:

| | | | |
|---|---|---|---|
| OK | Data type | 0 | temperature value |

Ambient Temp, LD:

| | | |
|---|---|---|
| OK | Data type | temperature value |

HQ core temp, LD:

| | | |
|---|---|---|
| OK | Data type | relative frequency relative |

Skin conductance, LD:

| | | |
|---|---|---|
| OK | Data type | resistance indicator |

Accelerometer, LD:

| | | | |
|---|---|---|---|
| OK | Data type = | Sub ID | vertical, horizontal etc. |

Accelerometer, HD:

| | | | |
|---|---|---|---|
| OK | Data type | Sub ID | arbitrary units, channel value |

External serial port pass through:

| | | | |
|---|---|---|---|
| NU | Data type | Sub ID | received ASCII byte |

Unassigned data type, may be used for future expansion

Data type =

Polar M32 Heart rate receiver, LD: (data types 8˜9)

| | | |
|---|---|---|
| OK | Data type = | instant received Heart Beat time |

ECG, HD: (data types 10˜11)

| | | |
|---|---|---|
| OK | Data type = | ECG voltage sample |

RIP Bands, HD: (data types 12˜15)

| | | |
|---|---|---|
| AX | Data type = | RIP oscillator counter value |

In this table: “OK” is a single bit field indicating likely data validity or invalidity; “AX” is a single bit field indicating to which of two RIP sensors the following data refers; “LSB” abbreviates “least significant bit”; and D0 (D7, D10, D11, and D12) labels the 0'th bit (7'th, 10'th, 11'th, and 12'th bits, respectively) of the data field.

Record formats are advantageously adapted to the means of physical transport. In the exemplary embodiment providing SPI-16 and LVTTL output, record length is preferably 16 bits. For most SPI-16 receivers, the 16 bits form a single frame. Alternately, a time delay may be inserted between the two halves of the 16 bit frames. ASCII data is preferably formatted into four hexadecimal characters separated by an ASCII space character (20H) or other idle character. Data from additional types of sensors can be accommodated by providing an “escape” record type that includes a field identifying the additional record types. Data that cannot be fit into the payload of a 16 bit record can be accommodated by record chaining. Preferably, a single set of record types is defined for a family of processing device embodiments with a particular embodiment of such a family transmitting only its needed record types.

In addition to standard outputs just described, alternative embodiments of the devices of this invention provide special outputs for certain data or diagnostic outputs from selected processing blocks. The exemplary embodiment provides serial diagnostic output from RIP processing blocks in the format described in Table 3.

TABLE 3
Exemplary record types and record formats

| | | | |
|---|---|---|---|
| BR | VF | SL | oscillator counter value |

Here, “BR” is a single bit field indicating a breath cycle is recognized; “VF” is a single bit field indicating that valid respiratory airflow (the time derivative of lung volume) is recognized; and “SL” is a single bit field indicating the sign of the slope of lung volume that has been determined.

These RIP diagnostic outputs can be displayed, stored and analyzed in order to validate RIP processing by the device, to verify its accuracy, and to explore changes to RIP processing firmware. For example, RIP counter values can be compared to concurrently respiratory measurement made by calibrated clinical tools in order to verify quantitative accuracy. The “BR”, “VF”, and “SL” bits reflect processing of counter values to determine breath occurrences, rates, and other summary respiratory indicators. Accuracy of this processing can be verified by comparing the values of these bits to concurrent RIP counter values. For example, “BR” or “SL” can be verified by direct comparison to a trace of lung volume (i.e., RIP counter values).

Physical and Hardware Configurations

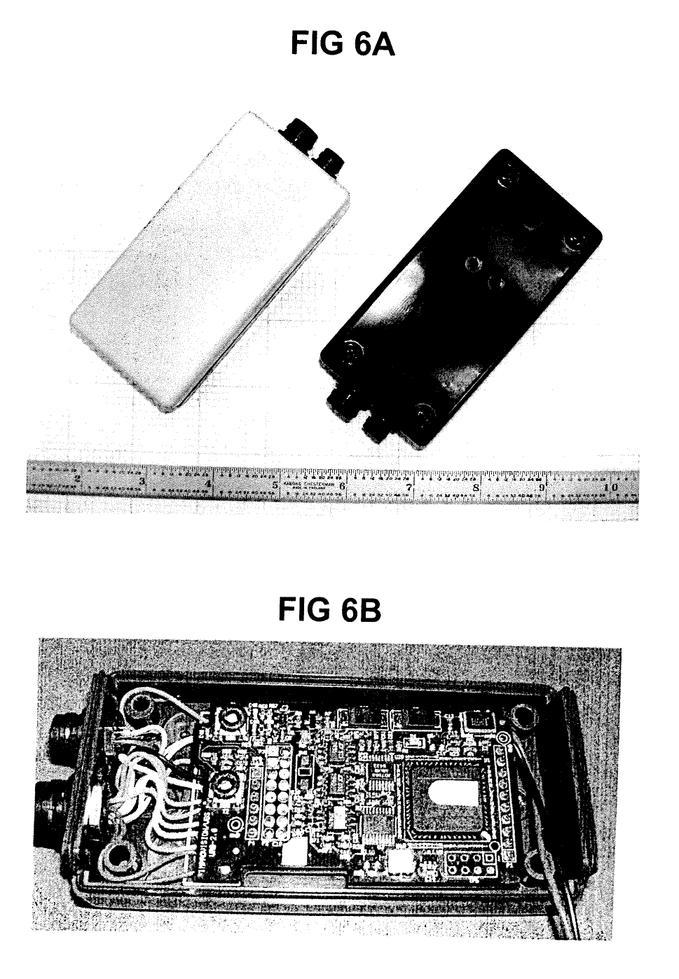

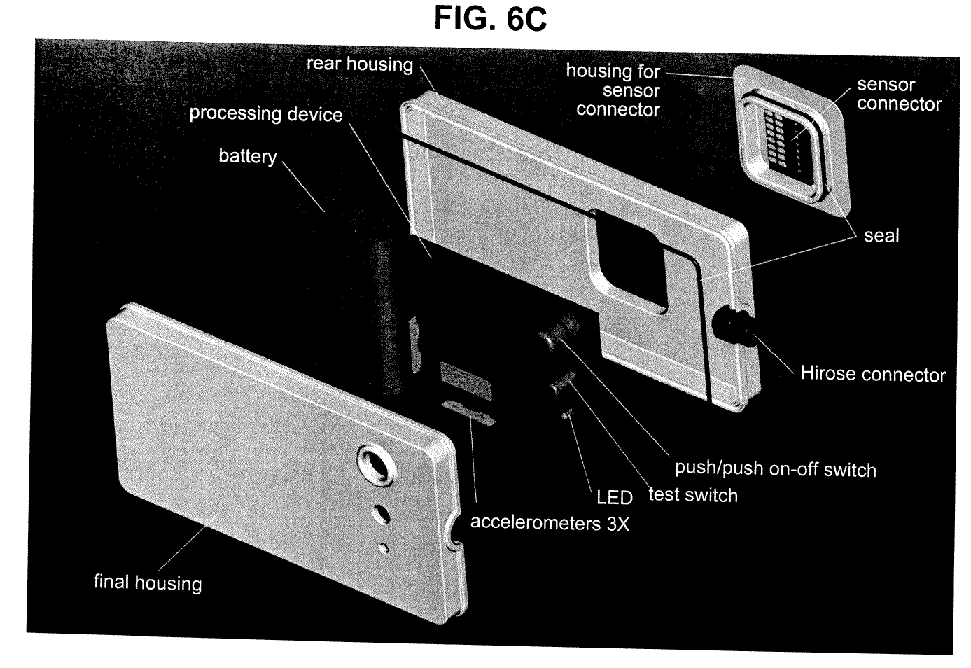

As already described with reference to FIG. 1, boards and devices of this invention are useful components of a wide range of different physiological monitoring systems. As illustrated with reference to FIGS. 6A-C, boards and devices of this invention can be configured in a wide variety of different physical and hardware configurations suitable for different physiological monitoring systems.

FIG. 6A illustrates a complete package housing the exemplary device described herein. External connectors are provided to external systems such as a monitoring subsystems having one or more sensors, or a communications subsystem, or a data recorded subsystem (generically “hosts”). External connectors can also be provided to the device's diagnostic ports and test points (if any), to JTAG connectors; or to external power or battery chargers (if any).

FIG. 6B illustrates a physical configuration of an implementation of the exemplary device as a single processing board in the exemplary package and housing of FIG. 6A. FIG. 6C illustrates an alternative physical implementation which includes a battery.

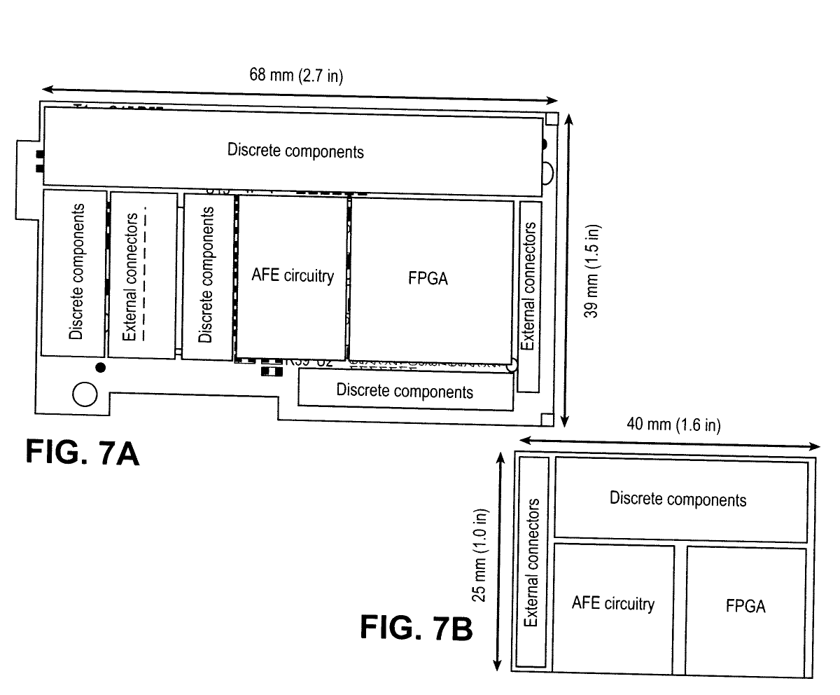

FIG. 7A generally illustrates the board configuration of FIG. 6B. Certain of the internal connectors permit stacked arrangements (mezzanine configurations) or side by side arrangements (daughter board configurations). FIG. 7B illustrates a possible physical implementation of an exemplary device for a reduced number of sensors. Even smaller configuration can be implemented for one or two sensors—e.g., one RIP band, one ECG lead

The devices and boards of this inventions can function allowing standalone, be a daughter board to a host device, be connected to external devices (e.g., a data recoding or communication module), configured with mezzanine boards, and so forth.

Aspects of this invention therefore include but are not limited to the following: The FPGA based architecture provides for function replication and expansion without impact on performance. This is not possible with CPU based designs. This is best exemplified by the 2 identical RIP functions for RC and AB bands. Most circuits (hardware AFEs and internal FPGA) include self validation or “confidence values” derived in real time. The device achieves low processing overhead or loss of CPU power, true real time response to multiple sensor events (respiration, heart rate, position, activity, temp, etc) while processing each stream independently of the others. Recursive hardware processing is implemented, that is processing algorithms and processed data adapt and are modified in real time according to interrelated rule sets acting on recently observed values. Adaptive power consumption profile turns off hardware whenever possible to conserve battery power and increase battery life per charge (up to more than 200 hours). The RIP oscillator circuit design and method of use achieves low power consumption (800 uA), smooth RIP data, and a ability to detect cycles at 20 oscillator “clicks” (reflecting extremely minimal chest movement). Auto adaptivity to 1 or 2 RIP bands, and auto range centering of RIP signal on power up (or other calibration period). Auto calibration nature of “body position” so that the definition of “vertical” auto sensed on power up (or other calibration period). Optional redundant sensor use (2D and 3D accelerometers, polar and ECG HR derivation). This increases immunity to possible sensor failure or malfunction. In the case of HR this permits possible use with known equipment used in sports applications. Heart rate noise blocking and marking resulting in agile response (not averaging based) while removing data spikes caused by motion artifacts and electrode release. The device provides a fully flexible ECG data format (in terms of data rate and resolution) allowing a coarse to fine mode of use.

The preferred embodiments of the invention described above do not limit the scope of the invention, since these embodiments are illustrations of several preferred aspects of the invention. Any equivalent embodiments are intended to be within the scope of this invention.

A number of references are cited herein, the entire disclosures of which are incorporated herein, in their entirety, by reference for all purposes. Further, none of these references, regardless of how characterized above, is admitted as prior to the invention of the subject matter claimed herein.

### Further Embodiments of Confidence Value Determination

In a further embodiment, the device can generate a “confidence value” (CV) for respiration data which can be added as an extension of the data format generated in LD data mode as described above. A preferred method for generating a CV is now described.

The device integrates RIP oscillations over 20 mSec periods. Dual rank techniques are used to measure the average frequency in each sample with 8 Mhz time base, and the result is rounded to 13 bits providing a range of 8192 data points for RIP frequency change measurements. The RIP value is centered within this range during device power up initialization to avoid possible overflow or underflow conditions. The 20 mSec samples are processed by a digital filter (FIR, 255 taps), followed by a breath detector for purposes of deriving a binary waveform whose rising edges correspond to inspiration cycles. When sending LD data, transmitted words are the time of each detected inspiration cycle in 20 mSec. units. The device monitors the RIP band, and if properly functioning, delivers a “band OK” bit in each LD word. This bit reflects the conditions of a correct electrical connection of the band and reasonable frequency range of the associated RIP oscillator.

The breath detector is a state machine that examines data samples for time domain trends. Observations made to reveal small trend changes (“jiggles”) which are mostly present at high levels of activity such as running or pushups. The state machine discriminates these events so they do not count as valid breath cycles. In order to reduce false cycles in low activity levels (sitting), the state machine also monitors the RIP value trend and considers possible changes (real cycles and “jiggles”) only when the RIP value slope is above a pre determined 1^{st} derivative (“valid flow”) value. The goals of jiggle discrimination and shallow breath cycle detection are opposite and can force the state machine to favor one detection mode over another.

An optional feature determines a confidence value based on a time histogram (also called boxcar integration) of the valid flow event. The amount of time a determined indicator (VF) is set to false during a given time window indicates that the RIP value is not showing trend changes typical to normal breathing, e.g., during breath holding or apnea. Since reading to a low rate of 5 BPM or 12 sec. per cycle, the proposed boxcar integrator can monitor a window of up to 16 seconds (3.75 BPM).

Actual histogram implementation in the device FPGA can validate VF and accumulate the zero valued (20 mSec) samples count in one second bins. A separate FIFO state machine can keep a running sum of these bins for the recent 16 seconds. Finally, the 16 Sec. boxcar count can be converted to a 3 bit CV where 7 implies high CV and 0 implies worst case CV.

Since low CV can be associated with no detection of real breath cycles—CV lower than a pre-determined threshold can force the device to transmit an LD data word in which the cycle time can be invalid. Absolute boxcar count to CV value mapping and low CV threshold setting can be developed based on field testing.

Another optional feature computes an adaptive “jiggle discriminator” value (ADV) based on recent RIP activity. Here, recent min-max RIP values can be used to adjust the minimal value change (MVC) required to differentiate jiggles from real breath cycles (e.g., 5% of a typical “brisk or long walking” RIP signature as observed to date). While improving accurate detection of shallow or shallow and slow breath cycles, ADV use can increase the number of false detected cycles when rapid increase in activity levels occur (a sitting subject standing up and starting to run). Similarly, cycles can be missed when quickly dropping the activity level (observations show that this is less likely). Actual ADV implementation in the device FPGA can be based on storing RIP values as FIFO using BRAM and operating a swing sorter state machine on these values (where swing is defined as the min to max difference within the FIFO space). Three possible windows of 4/8/16 Sec. can be used for swing calculation. The determined swing can be mapped into a correction factor that can be added to the present MVC.

Selection of the time window used to determine swing, and the mapping of absolute swing value to MVC correction value can require field testing. Finally note that a further development can involve selective activation of the ADV based on present CV. Such development is not suggested for implementation at this phase, but should be considered in the future.

What is claimed is:

1. A device for processing signals from a plurality of physiological sensors comprising:
a plurality of functional digital, processing units that are configurable to perform steps including
receiving signals from the sensors;
processing the received signals to determine physiological information; and
multiplexing the determined physiological information in one or more output signals,
wherein, for the signals for two or more sensors, the steps of receiving and processing are performed by one or more processing units that operate concurrently and in parallel.

2. The device of claim 1 wherein, for one or more sensor signals, two or more of the associated processing units are operate in series as a processing pipeline.

3. The device of claim 1 wherein separate physiological information is derived from two or more sensor signals in a substantially simultaneous manner.

4. The device of claim 1 further comprising a field programmable gate array (FPGA).

5. The device of claim 4 wherein functional digital, processing units are configured by loading firmware prior to the sensor signal processing.

6. The device of claim 1 further comprising analog front end (AFE) circuitry for analog preprocessing of signals from analog sensors.

7. The device of claim 1 wherein some or all of the circuitry for processing a particular sensor signal is powered down if a signal is not being received from the particular sensor.

8. The device of claim 1 wherein some or all of the circuitry for processing a particular sensor signal is powered down for some or all of the time interval between signal samples received from the particular sensor.

9. The device of claim 1 wherein the functional digital, processing units are further configurable to process a signal from at least one of an accelerometer sensor, an ECG sensor, a heart rate sensor, a body temperature sensor, an electroencephalographic sensor, and a sound sensor.

10. The device of claim 1 further comprising one or more sensors sensitive to status of the device; and wherein the output signal further comprises device status information determined from the device sensors.

11. The device of claim 1 wherein the concurrent processing further comprises determining status of signals from one or more sensors; and wherein the output signal further comprises the determined sensor-signal status information.

12. A method for processing signals from a plurality of physiological sensors comprising:
receiving concurrently signals from two or more of the sensors;
receiving signals from the sensors;
processing the received signals to determine physiological information; and
multiplexing the determined physiological information in one or more output signals,
wherein the receiving and processing proceed concurrently and in parallel for the signals from two or more of the sensors.

13. The method of claim 12 wherein, for one or more sensor signals, concurrent processing comprises two or more steps arranged in series and occurring simultaneously as a processing pipeline.

14. The method of claim 12 wherein processing of at least one sensor signal does not substantially delay processing of other sensor signals.

15. The method of claim 12 further comprising receiving signals sensitive to status of the device; and wherein the output signal further comprises device status information.

16. The method of claim 12 wherein sensor comprise one or more of an inductive plethysmographic (IP) sensor, a respiratory IP (RIP) sensor, an accelerometer sensor, an ECG sensor, a heart rate sensor, a body temperature sensor, an electroencephalographic sensor, and a sound sensor.

17. The method of claim 12 further comprising adjusting the processing of one or more sensor signals according to calibration information, the calibration information being determined during a previous calibration period prior the processing.

18. The method of claim 17 further comprising determining the calibration information for two or more sensors during the same calibration period.

19. The method of claim 17 wherein the output signal comprises one of more calibrated sensor signals.

20. The method of claim 17 wherein the calibration information for one or more IP sensors comprises a signal output range, and wherein the IP sensor signals are centered within the output range.

21. The method of claim 17 wherein the calibration information for one or more accelerometer sensors comprises a vertical reference value.

22. The method of claim 12 further comprising receiving signals from two or more IP sensors sensitive to the subject's respiration (RIP), wherein respiratory information concerning the subject is determined in part by combining the RIP signals.

23. A system for physiological monitoring of a subject comprising:
a plurality of physiological sensors including one or more inductive plethysmographic (IP) sensor; and
a device for processing signals from the plurality of sensors comprising a plurality of functional digital, processing units that are configurable to perform steps including
receiving signals from the IP sensor and at one or more other sensor;
processing the received signals to determine physiological information concerning the subject; and
multiplexing the determined physiological information in one or more output signals,
wherein, for the signals for the IP sensor and one or more other sensors, the steps of receiving and processing are performed by one or more processing units that operate concurrently and in parallel.

24. The system of claim 23 further comprising one or more sensors selected from an accelerometer sensor, an ECG sensor, a heart rate sensor, a body temperature sensor, an electroencephalographic sensor, and a sound sensor; and
wherein the functional digital, processing units are configurable to process signals from the one or more sensors.

25. The system of claim 23 further comprising a plurality of IP sensors sensitive to the subject's respiration (RIP sensor); and
wherein the concurrent processing further comprises combining signals from at least two RIP sensors to determine respiratory information concerning the subject.

26. The system of claim 23 further comprising a housing.

27. The system of claim 23 further comprising a battery for powering the device.

28. The system of claim 23 further comprising a wireless communication unit for wirelessly communicating the physiological information remotely from the system.

29. The system of claim 23 further comprising a data recording unit for recording at recording the physiological information locally at the system.

30. The system of claim 23 wherein the subject comprises a mammal.
